(19) United States
(12) Patent Application Publication
(10) Pub. No.: US 2026/0345270 A1
(43) Pub. Date: Nov. 5, 2026

(54) SURGICAL ROBOTIC SYSTEM AND METHOD FOR VELOCITY-BASED FORCE FEEDBACK IN HANDLE CONTROLLER

(71) Applicant: Covidien LP, Mansfield, MA (US)

(72) Inventors: Christopher T. Tschudy, Arvada, CO (US); Connor Roberts, Arvada, CO (US); Haralambos P. Apostolopoulos, Westminster, CO (US)

(21) Appl. No.: 19/489,016

(22) PCT Filed: Jun. 3, 2024
(86) PCT No.: PCT/IB2024/055413

§ 371 Date: Dec. 1, 2025

Related U.S. Application Data
(60) Provisional application No. 63/471,791, filed on Jun. 8, 2023.

(51) Int. Cl. A61B 34/37; A61B 17/28; A61B 34/00; A61B 34/30; A61B 90/00

(52) U.S. Cl. CPC A61B 34/37; A61B 17/282; A61B 34/76; A61B 34/77; A61B 90/06; A61B 2034/305; A61B 2090/064

(57) ABSTRACT

A surgical robotic system may include a robotic arm and an instrument drive unit. The system also includes an instrument coupled to and actuatable by the instrument drive unit. The instrument includes a first jaw member and a second jaw member, where at least one of the jaw members is movable relative to the other jaw member from an open to a closed jaw position. The system further includes a surgeon console having a handle controller with a handle and a paddle pivotably movable relative to the handle from an open paddle position to a paddle closed position. Movement of the paddle is used to control movement of at least one of the first or second jaw members. The handle controller also includes a feedback motor configured to provide force feedback to the paddle based on a position and a velocity of the paddle relative to the handle.

### CROSS-REFERENCE TO RELATED APPLICATION

This application claims the benefit of U.S. Provisional Patent Application Ser. No. 63/471,791, filed Jun. 8, 2023, the entire content of which is incorporated herein by reference.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. Surgical robotic systems are used with a variety of jawed surgical instruments, such as graspers, cutters, electrosurgical vessel sealers, etc.

### SUMMARY

According to one embodiment of the present disclosure, a surgical robotic system is disclosed. The surgical robotic system may include a robotic arm and an instrument drive unit. The system also includes an instrument coupled to and actuatable by the instrument drive unit. The instrument includes a first jaw member and a second jaw member, where at least one of the first or second jaw members is movable relative to the other of the first or second jaw members from an open jaw position to a closed jaw position. The system further includes a surgeon console having a handle controller with a handle and a paddle pivotably movable relative to the handle from an open paddle position to a paddle closed position. Movement of the paddle is used to control movement of at least one of the first or second jaw members. The handle controller also includes a feedback motor configured to provide force feedback to the paddle based on a position and a velocity of the paddle relative to the handle.

Implementations of the above embodiment may include one or more of the following features. According to one aspect of the above embodiment, the feedback motor is configured to measure the position and the velocity of the paddle. The surgical robotic system may include: a storage device storing a force feedback profile. The surgical robotic system may include a controller configured to calculate the force feedback based on the position and the velocity of the paddle from the force feedback profile; and control the feedback motor to provide the calculated force feedback. The controller is further configured to adjust the force feedback profile based on the velocity of the paddle. The surgical robotic system may include: a storage device storing a plurality of force feedback profiles. The surgical robotic system may include a controller configured to select a force feedback profile from the plurality of force feedback profiles based on the velocity of the paddle. The controller is further configured to calculate the force feedback based on the position of the paddle from the selected force feedback profile; and control the feedback motor to provide the calculated force feedback. The instrument may further include a coupler driven by a motor held by the robotic arm; a drive rod longitudinally movable by the coupler configured to move at least one of the first or second jaw members relative to the other of the first or second jaw members; and a spring compressed by the coupler during movement of the drive rod.

According to another embodiment of the present disclosure, a method for controlling a surgical robotic system is disclosed. The method may include actuating an instrument coupled to and actuatable by an instrument drive unit, the instrument including a first jaw member and a second jaw member. At least one of the first or second jaw members movable relative to the other of the first or second jaw members from an open jaw position to a closed jaw position. The method includes receiving an input from a surgeon console having a handle controller with a handle and a paddle pivotably movable relative to the handle from an open paddle position to a paddle closed position, where the input includes movement of the paddle. The method further includes controlling movement of at least one of the first or second jaw members by the instrument drive unit based on the input. The method additionally includes providing force feedback to the paddle via a feedback motor based on a position and a velocity of the paddle relative to the handle.

Implementations of the above embodiment may include one or more of the following features. According to one aspect of the above embodiment, the method may include measuring the position and the velocity of the paddle through the feedback motor. The method may further include storing a force feedback profile at a storage device. The method may additionally include calculating, at a controller, the force feedback based on the position and the velocity of the paddle from the force feedback profile and controlling the feedback motor to provide the calculated force feedback. The method may also include adjusting the force feedback profile based on the velocity of the paddle. The method may further include storing a plurality of force feedback profiles at a storage device and selecting, at a controller, a force feedback profile from the plurality of force feedback profiles based on the velocity of the paddle. The method may additionally include calculating the force feedback based on the position of the paddle from the selected force feedback profile and controlling the feedback motor to provide the calculated force feedback.

According to a further embodiment of the present disclosure, a surgical robotic system is disclosed. The surgical robotic system includes a robotic arm and an instrument drive unit. The system also includes an instrument coupled to and actuatable by the instrument drive unit. The instrument also includes a first jaw member and a second jaw member, where at least one of the first or second jaw members is movable relative to the other of the first or second jaw members from an open jaw position to a closed jaw position. The system also includes a surgeon console having a handle controller with a handle and a paddle pivotably movable relative to the handle from an open paddle position to a paddle closed position. Movement of the paddle is used to control movement of at least one of the first or second jaw members. The handle controller also includes a feedback motor configured to measure a position and a velocity of the paddle relative to the handle. The system also includes a storage device storing a plurality of force feedback profiles. The system additionally includes a controller configured to select a force feedback profile from the plurality of force feedback profiles based on the velocity of the paddle and control the feedback motor to provide force feedback to the paddle based on the position and the velocity of the paddle relative to the handle.

Implementations of the above embodiment may include one or more of the following features. According to one aspect of the above embodiment, the controller is further configured to: calculate the force feedback based on the position of the paddle from the selected force feedback profile and control the feedback motor to provide the calculated force feedback. Each force feedback profile may include a plurality of force feedback values each of which corresponds to a position value of the paddle.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a perspective view of a surgical instrument provided in accordance with the present disclosure configured for mounting on a robotic arm of a robotic surgical system;

FIG. 8 is a front, perspective view of a proximal portion of the surgical instrument of FIG. 7 with an outer shell removed;

FIG. 9 is a rear, perspective view of the proximal portion of the surgical instrument of FIG. 7 with the outer shell removed;

FIG. 10 is a front, perspective view of the proximal portion of the surgical instrument of FIG. 7 with the outer shell and additional internal components removed;

FIG. 11 is a perspective view of a surgical instrument according to another embodiment of the present disclosure;

FIGS. 12A and B are perspective views of an end effector of the surgical instrument of FIG. 11 in open and closed configurations;

FIG. 13 is a perspective view of a handle controller according to one embodiment of the present disclosure;

FIGS. 14 and 15 are partially disassembled views of the handle controller according to one embodiment of the present disclosure;

FIG. 16 is a flow chart of a method for velocity-based force feedback in the handle controller according to one embodiment of the present disclosure; and

FIG. 17 shows a plurality of velocity-based force feedback plots according to one embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives operator input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands to control one or more actuators of the robotic arm, which would, in turn, move the robotic arm and the instrument in response to the movement commands.

The instrument is a forceps having a pair of opposing jaws with one or both of the jaws being movable relative to each other. In embodiments, the forceps may be electrosurgical forceps configured to seal tissue, e.g., blood vessel(s). The jaws are actuated by a drive rod that is engaged by a spring to provide for a consistent pressure applied by the opposing jaws on the tissue.

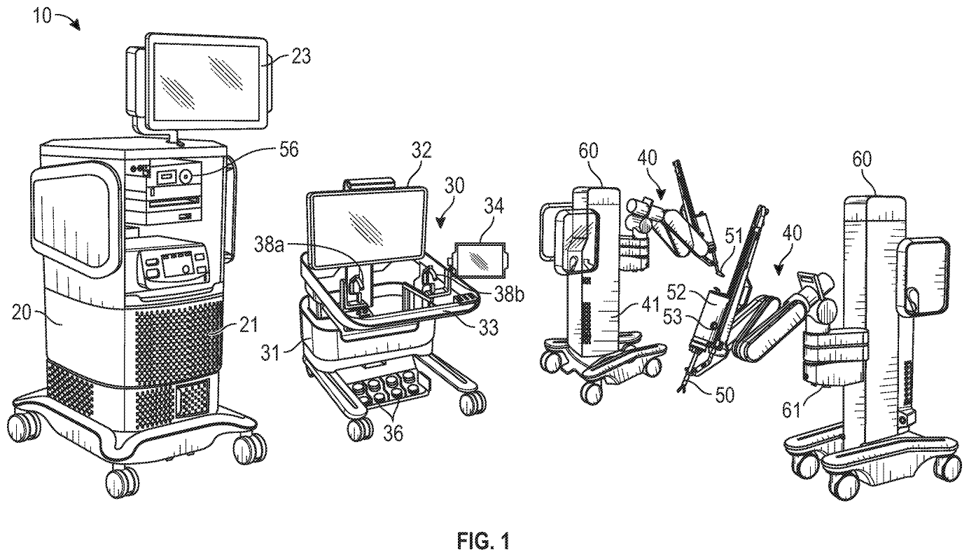

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of operator interface devices, such as foot pedals 36 and a pair of handle controllers 38a and 38b which are used by an operator to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the handle controllers 38a and 38b, repositioning camera movement and electrosurgical activation/deactivation. In particular, the foot pedals 36 may be used to perform a clutching action on the handle controllers 38a and 38b. Clutching is initiated by pressing one of the foot pedals 36, which disconnects (i.e., prevents movement inputs) the handle controllers 38a and/or 38b from the robotic arm 40 and corresponding instrument 50 or camera 51 attached thereto. This allows the operator to reposition the handle controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

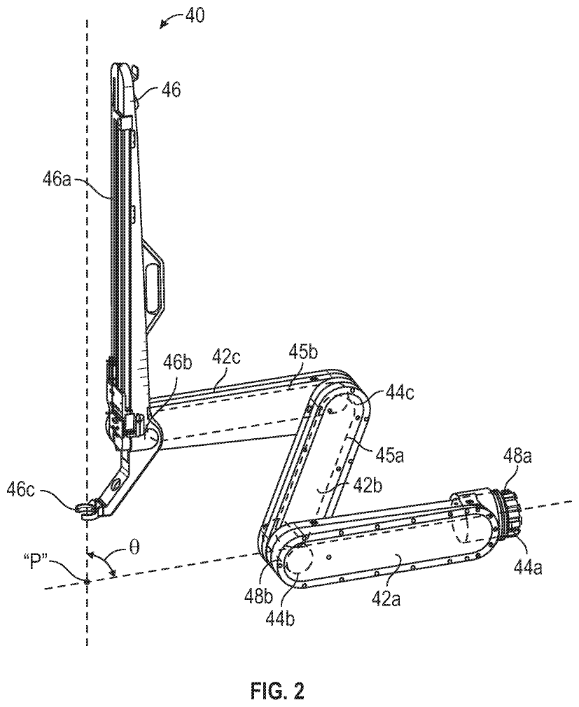

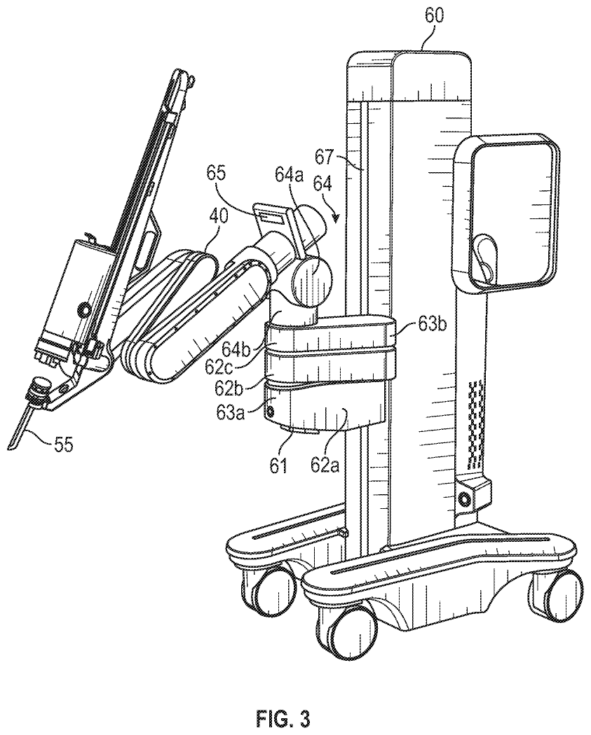

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. Joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 65 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48 b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 140 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The operator may press one or more of the buttons 53 to move the component associated with the button 53.

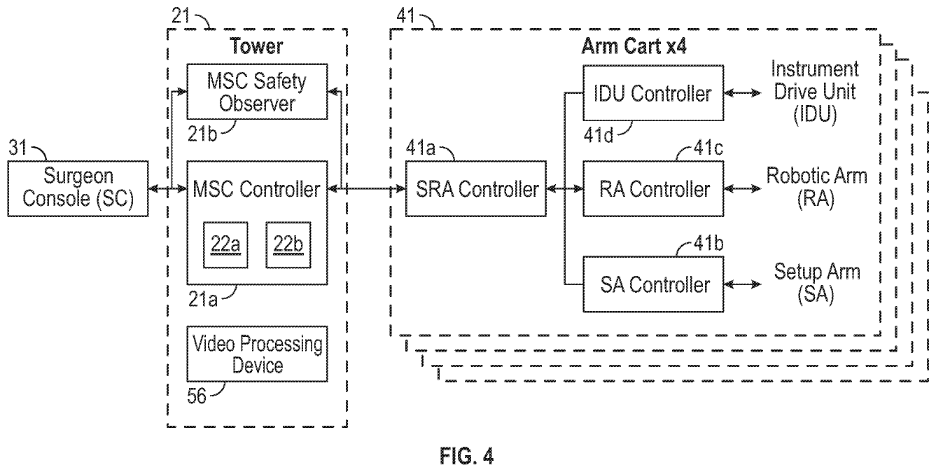

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by an operator. The joints 63a and 63b and the rotatable base 64 include brakes that are disengaged by the operator to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

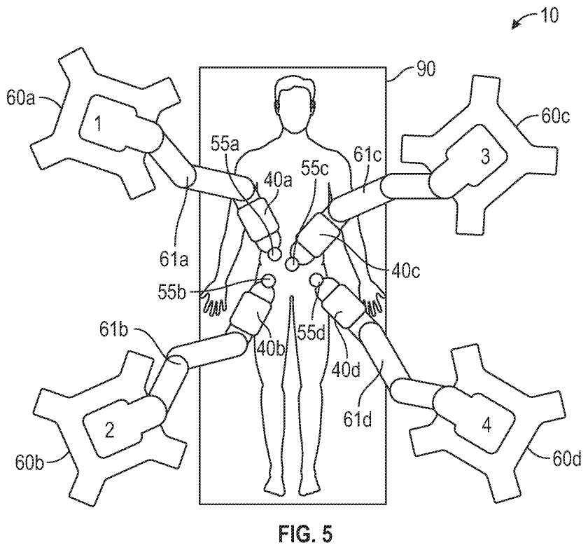

With reference to FIG. 5, the surgical robotic system 10 is set up around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

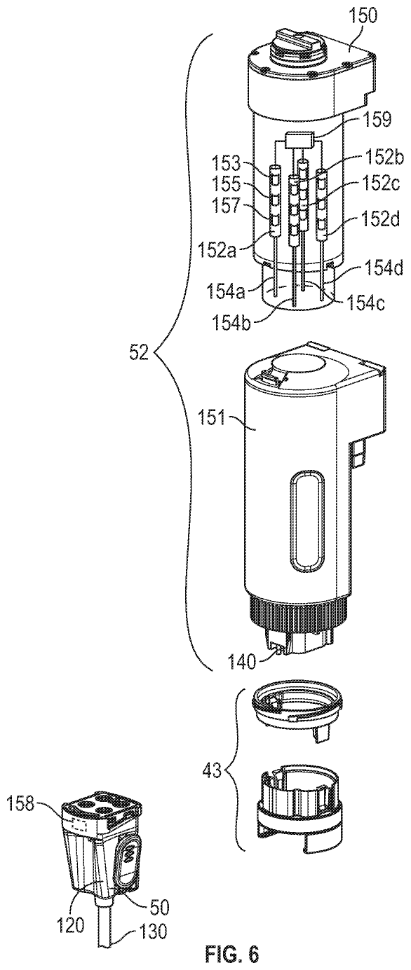

With reference to FIG. 6, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

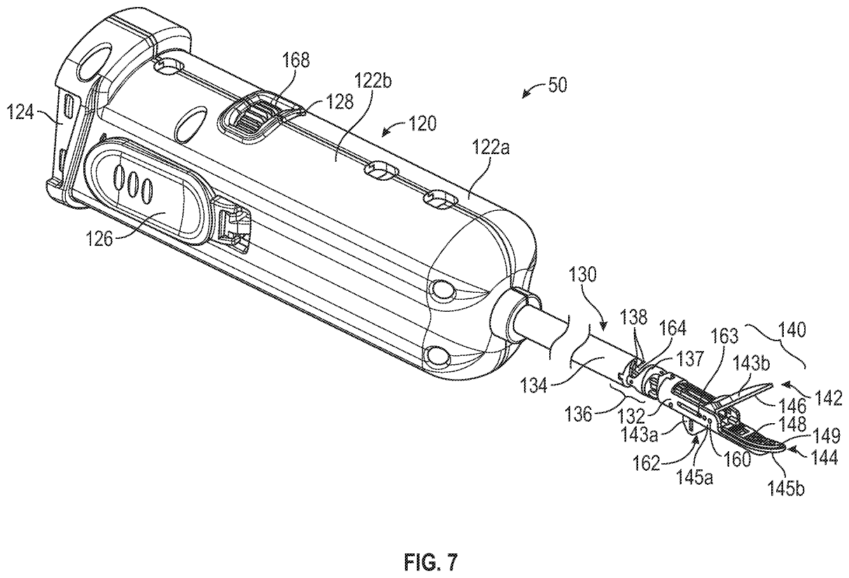

The IDU 52 includes a motor pack 150 and a sterile barrier housing 151. Motor pack 150 includes motors 152a, 152b, 152c, 152d for controlling various operations of the instrument 50. The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50. The instrument 50 is configured to transfer rotational forces/movement supplied by the IDU 52 (e.g., via the motors 152a, 152b, 152c, 152d of the motor pack 150) into longitudinal movement or translation of the cables or drive shafts to effect various functions of an end effector assembly 140 (FIG. 7).

Each of the motors 152a, 152b, 152c, 152d includes a current sensor 153, a torque sensor 155, and an encoder sensor 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The encoder 157 may be any device that provides a sensor signal indicative of the number of rotations of the motor 152a, such as a mechanical encoder or an optical encoder. Parameters which are measured and/or determined by the encoder 157 may include speed, distance, revolutions per minute, position, and the like. The sensor signals from sensors 153, 155, 157 are transmitted to the IDU controller 41d, which then controls the motors 152a, 152b, 152c, 152d based on the sensor signals. In particular, the motors 152a, 152b, 152c, 152d are controlled by an actuator controller 159, which controls torque output by, and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

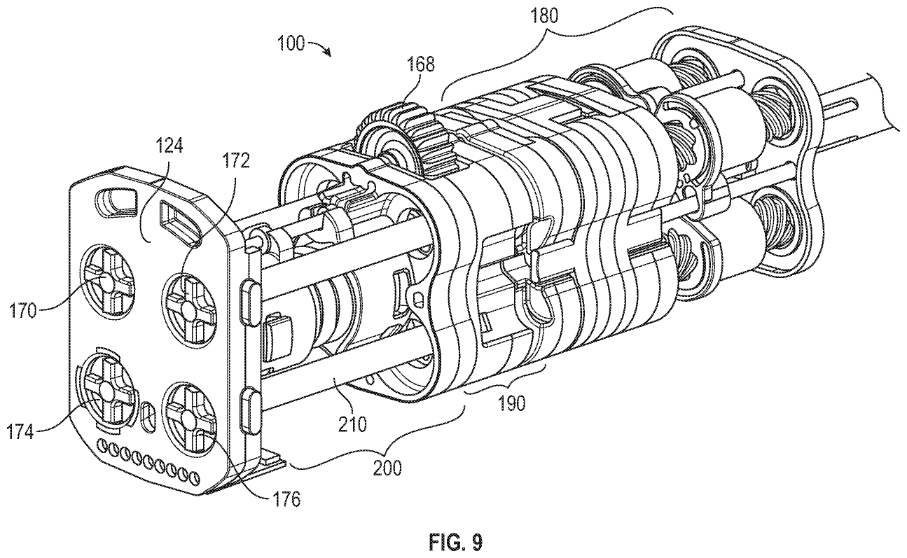

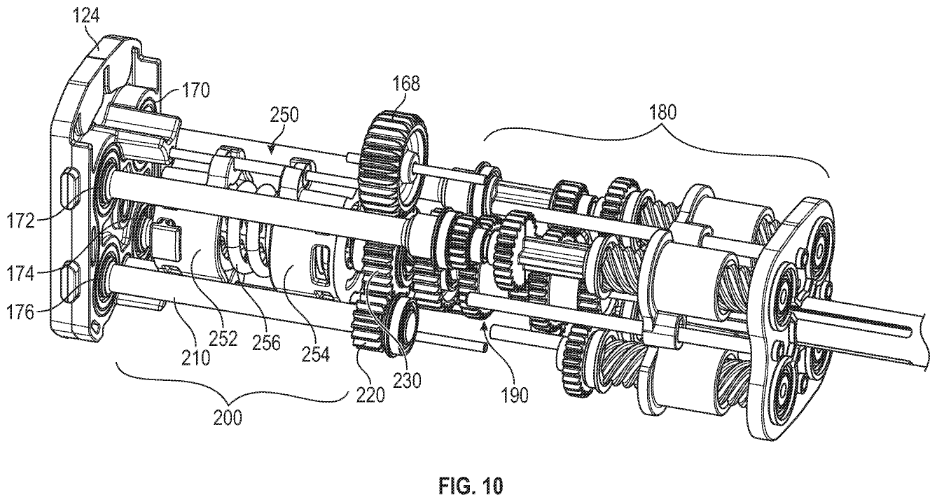

Referring to FIGS. 6-10, the instrument 50 includes the housing 120, a shaft 130 extending distally from housing 120, and end effector assembly 140 extending distally from shaft 130. A gearbox assembly 100 disposed within housing 120 and operably associated with end effector assembly 140. Housing 120 of instrument 50 is configured to selectively couple to IDU 52 of robotic, to enable motors 152a, 152b, 152c, 152d of IDU 52 to operate the end effector assembly 140 of the instrument 50. Housing 120 of instrument 50 supports a drive assembly that is mechanically actuated by the motors 152a, 152b, 152c, 152d of the IDU 52. Drive assembly of instrument 50 may include any suitable electrical and/or mechanical component to effectuate driving force/movement.

Instrument 50 is described herein as an articulating electrosurgical forceps configured for use with the robotic surgical system 10. However, the aspects and features of instrument 50 provided in accordance with the present disclosure, detailed below, are equally applicable for use with other suitable surgical instruments and/or in other suitable surgical systems.

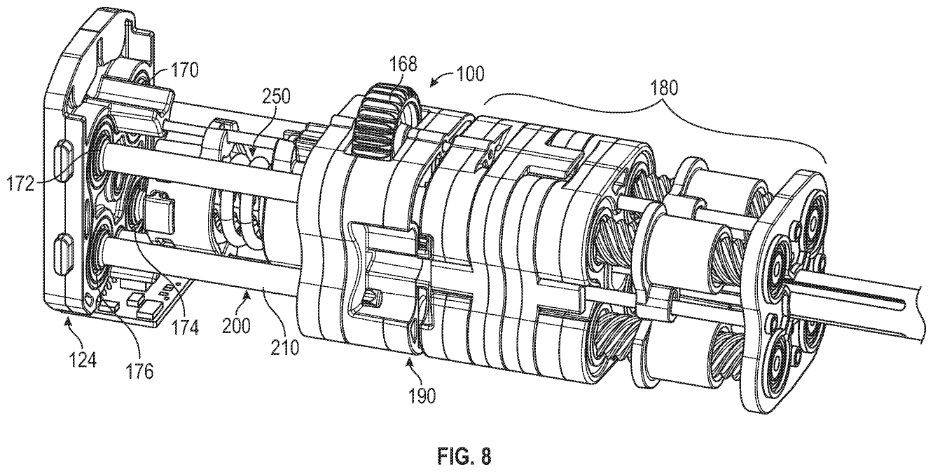

The housing 120 of instrument 50 includes first and second body portion 122a, 122b and a proximal face plate 124 that cooperate to enclose gearbox assembly 100 therein. Proximal face plate 124 includes apertures defined with couplers 170, 172, 174, 176 of gearbox assembly 100 extending through proximal face plate 124 (FIG. 8). A pair of latch levers 126 (only one of which is illustrated in FIG. 7) extend outwardly from opposing sides of housing 120 and enable releasable engagement of housing 120 with the IDU 52 of the robotic arm 40. An aperture 128 defined through housing 120 permits thumbwheel 168 to extend therethrough to enable manual manipulation of thumbwheel 168 from the exterior of housing 120 to permit manual opening and closing of end effector assembly 140.

Shaft 130 of instrument 50 includes a distal segment 132, a proximal segment 134, and an articulating section 136 disposed between the distal and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal segment 132 of shaft 130 at the distal ends thereof and extend proximally from distal segment 132 of shaft 130, through articulating section 136 of shaft 130 and proximal segment 134 of shaft 130, and into housing 120, wherein articulation cables 138 operably couple with an articulation sub-assembly 180 of gearbox assembly 100 to enable selective articulation of distal segment 132 (and, thus end effector assembly 140) relative to proximal segment 134 and housing 120, e.g., about at least two axes of articulation (e.g., yaw and pitch articulation). Articulation cables 138 may be arranged in a generally rectangular configuration, although other suitable configurations are also contemplated.

Articulation of end effector assembly 140 relative to proximal segment 134 of shaft 130, is accomplished by actuation of pair of cables 138. More specifically, in order to pitch end effector assembly 140, the upper pair of cables 138 are actuated while the lower pair of cables 138 are actuated relative to one another but in opposite manner relative to the upper pair of cables 138. With respect to yaw articulation, the right pair of cables 138 are actuated while the left pair of cables 138 are actuated but in opposite manner relative to the right pair of cables 138.

Continuing with reference to FIG. 7, end effector assembly 140 includes first and second jaw members 142, 144, respectively. Each jaw member 142, 144 includes a proximal flange portion 143a, 145a and a distal body portion 143b, 145b, respectively. Distal body portions 143b, 145b define opposed tissue-contacting surfaces 146, 148, respectively. Proximal flange portions 143a, 145a are pivotably coupled to one another about a pivot 160 and are operably coupled to one another via a cam-slot assembly 162 including a cam pin 163 slidably received within cam slots defined within the proximal flange portion 143a, 145a of at least one of the jaw members 142, 144, respectively, to enable pivoting of jaw member 142 relative to jaw member 144 and distal segment 132 of shaft 130 between a spaced-apart position (e.g., an open position of end effector assembly 140) and an approximated position (e.g. a closed position of end effector assembly 140) for grasping tissue between tissue-contacting surfaces 146, 148. As an alternative to this unilateral configuration, a bilateral configuration may be provided whereby both jaw members 142, 144 are pivotable relative to one another and distal segment 132 of shaft 130.

In embodiments, longitudinally extending knife channels 149 (only knife channel 149 of jaw member 144 is illustrated; the knife channel of jaw member 142 is similarly configured) are defined through tissue-contacting surfaces 146, 148, respectively, of jaw members 142, 144. In such embodiments, a knife assembly including a knife tube (not shown) extending from housing 120 through shaft 130 to end effector assembly 140 and a knife blade (not shown) disposed within end effector assembly 140 between jaw members 142, 144 is provided to enable cutting of tissue grasped between tissue-contacting surfaces 146, 148 of jaw members 142, 144, respectively. Knife tube (not shown) is operably coupled to a knife drive sub-assembly 190 of gearbox assembly 100 at a proximal end thereof to enable selective actuation thereof to, in turn, reciprocate the knife blade (not shown) between jaw members 142, 144 to cut tissue grasped between tissue-contacting surfaces 146, 148.

Referring to FIG. 7, a drive rod 164 is operably coupled to cam-slot assembly 162 of end effector assembly 140, e.g., engaged with the cam pin 163 thereof, such that longitudinal actuation of drive rod 164 pivots jaw member 142 relative to jaw member 144 between the spaced-apart and approximated positions. More specifically, urging drive rod 164 proximally pivots jaw member 142 relative to jaw member 144 towards the approximated or closed position while urging drive rod 164 distally pivots jaw member 142 relative to jaw member 144 towards the spaced-apart, open position. However, other suitable mechanisms and/or configurations for pivoting jaw member 142 relative to jaw member 144 between the spaced-apart and approximated positions in response to selective actuation of drive rod 164 are also contemplated. Drive rod 164 extends proximally from end effector assembly 140 through shaft 130 and into housing 120 wherein drive rod 164 is operably coupled with a jaw drive sub-assembly 200 of gearbox assembly 100 to enable selective actuation of end effector assembly 140 to grasp tissue therebetween and apply a closure force within an appropriate force range.

Tissue-contacting surfaces 146, 148 of jaw members 142, 144, respectively, are at least partially formed from an electrically conductive material and are energizable to different potentials to enable the conduction of electrical energy through tissue grasped therebetween, although tissue-contacting surfaces 146, 148 may alternatively be configured to supply any suitable energy, e.g., thermal, microwave, light, ultrasonic, ultrasound, etc., through tissue grasped therebetween for energy-based tissue treatment. Instrument 50 defines a conductive pathway (not shown) through housing 120 and shaft 130 to end effector assembly 140 that may include lead wires, contacts, and/or electrically-conductive components to enable electrical connection of tissue-contacting surfaces 146, 148 of jaw members 142, 144, respectively, to an energy source (not shown), e.g., an electrosurgical generator, for supplying energy to tissue-contacting surfaces 146, 148 to treat, e.g., seal, tissue grasped between tissue-contacting surfaces 146, 148.

With reference to FIGS. 8-10, the gearbox assembly 100 is disposed within housing 120 and includes an articulation sub-assembly 180, a knife drive sub-assembly 190, and a jaw drive sub-assembly 200. Articulation sub-assembly 180 is operably coupled between first and second couplers 170, 172, respectively, of gearbox assembly 100 and articulation cables 138 (FIG. 7) such that, upon receipt of appropriate inputs into first and/or second couplers 170, 172, articulation sub-assembly 180 manipulates cables 138 (FIG. 7) to articulate end effector assembly 140 in a desired direction, e.g., to pitch and/or yaw end effector assembly 140.

Knife drive sub-assembly 190 is operably coupled between third coupler 174 of gearbox assembly 100 and knife tube (not shown) such that, upon receipt of appropriate input into third coupler 174, knife drive sub-assembly 190 manipulates knife tube to reciprocate the knife blade (not shown) between jaw members 142, 144 to cut tissue grasped between tissue-contacting surfaces 146, 148.

Jaw drive sub-assembly 200, as detailed below, is operably coupled between fourth coupler 176 of gearbox assembly 100 and drive rod 164 such that, upon receipt of appropriate input into fourth coupler 176, jaw drive sub-assembly 200 pivots jaw members 142, 144 between the spaced-apart and approximated positions to grasp tissue therebetween and apply a closure force within an appropriate closure force range.

Gearbox assembly 100 is configured to operably interface with the IDU 52 when instrument 50 is mounted on robotic surgical system 10. That is, the motors 152a, 152b, 152c, 152d of IDU 52 selectively actuate couplers 170-176 of gearbox assembly 100 to articulate end effector assembly 140, grasp tissue between jaw members 142, 144, and/or cut tissue grasped between jaw members 142, 144. However, it is also contemplated that gearbox assembly 100 be configured to interface with any other suitable surgical system, e.g., a manual surgical handle, a powered surgical handle, etc.

With reference to FIGS. 8-10, jaw drive sub-assembly 200 of gearbox assembly 100 is shown generally including an input shaft 210, an input gear 220, a drive gear 230, the thumbwheel 168, a spring force assembly 250, and the drive rod assembly 164. The spring force assembly 250 includes a proximal hub 252, a distal hub 254, and a compression spring 256.

Compression spring 256 is disposed between proximal and distal hubs 252, 254 with a proximal portion thereof disposed within a cavity of proximal hub 252 and a distal portion thereof disposed within a cavity of distal hub 254. At least a portion of compression spring 256 is disposed about and/or configured to receive a portion of lead screw of drive gear 230 therethrough.

During use, jaw members 142, 144 are initially disposed in the spaced-apart position and, correspondingly, proximal and distal hubs 252, 254 are disposed in a distal-most position such drive rod 164 is disposed in a distal-most position. Further, in this position, compression spring 256 is disposed in a least-compressed condition; although even in the least-compressed condition, compression spring 256 may be partially compressed due to the retention of compression spring 256 in a pre-compressed configuration between proximal and distal hubs 252, 254.

In response to an input to close end effector assembly 140, e.g., rotational input to fourth (i.e., jaw) coupler 176 or a manual rotation of the thumbwheel 168, drive shaft 210 is rotated to thereby rotate input gear 220 which, in turn, rotates drive gear 230 such that distal hub 254 is translated proximally towards proximal hub 252. Proximal translation of distal hub 254 urges distal hub 254 against compression spring 256. Initially, where forces resisting approximation of jaw members 142, 144 are below a threshold corresponding to the spring value of compression spring 256, the closure force applied by jaw members 142, 144 is relatively low such that the urging of distal hub 254 proximally against compression spring 256 urges compression spring 256 proximally which, in turn, moves drive rod 164 proximally to pivot jaw member 142 relative to jaw member 144 from the spaced-apart position towards the approximated position to grasp tissue therebetween.

Upon further approximation of jaw members 142, 144 to grasp tissue therebetween, the forces resisting approximation of jaw members 142, 144, e.g., tissue resisting compression, may reach the threshold and, thus the closure force applied by jaw members 142, 144 may reach a corresponding threshold. In order to maintain the closure force applied by jaw members 142, 144 within a closure pressure range such as, for example, from about 3 kg/cm² to about 16 kg/cm², application of further closure force by jaw members 142, 144 is inhibited beyond this point despite further rotational input to fourth coupler 176. Once the threshold has been reached, further rotational input to fourth coupler 176 rotates drive shaft 210, input gear 220, and drive gear 230 to translate distal hub 254 further proximally into compression spring 256. However, rather than compression spring 256 urging proximal hub 252 further proximally to continue approximation of jaw members 142, 144 and increase the closure force applied therebetween, compression spring 256 is compressed, enabling proximal hub 252 and, thus, drive rod 164 to remain in position, thus inhibiting application of additional closure force between jaw members 142, 144. Operation of the instrument 50 and its components, including the compression spring 256, is described in more detail in U.S. Patent Application Publication No. 2020/0237453, filed on Jan. 29, 2019, the entire contents of which are incorporated by reference herein.

The surgical instrument 50 also includes a storage device 158 (FIG. 6). The storage device 158 includes non-volatile storage medium (e.g., EEPROM) that is configured to store any data pertaining to the surgical instrument 50, including but not limited to, usage count, identification information, model number, serial number, calibration data, and the like. In embodiments, the data may be encrypted and is only decryptable by the IDU controller 41d. The data may also be used by the IDU controller 41d to authenticate the surgical instrument 50. The storage device 158 may be configured in read only or read/write modes, allowing the IDU controller 41d to read as well as write data onto the storage device 158.

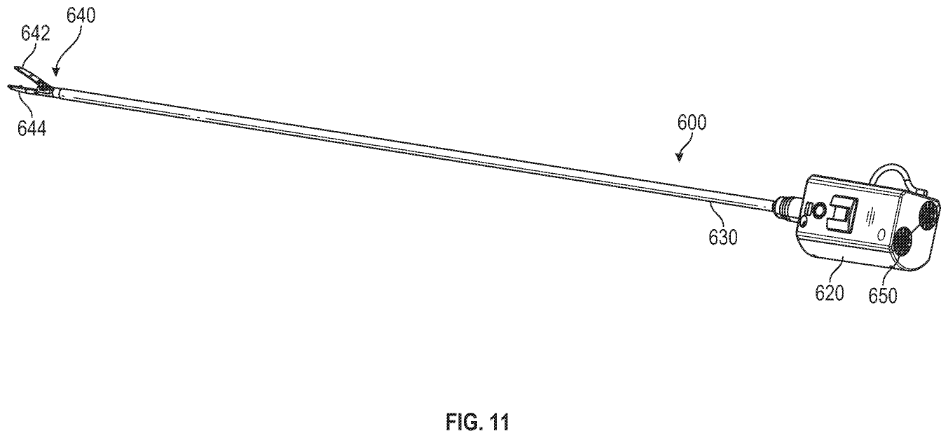

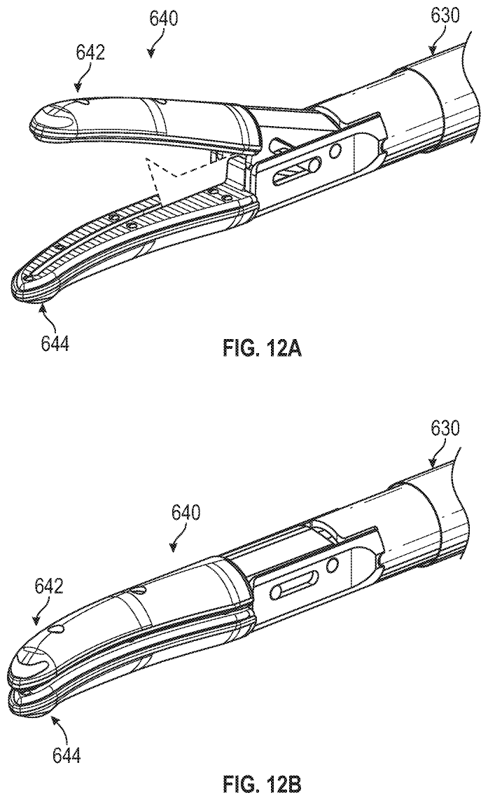

The disclosed system and method of force feedback may be applied to any spring-loaded jawed instrument, such as an instrument 600 of FIG. 11, which is similar to the instrument 50 with some variations, such as lack of any articulation joints. This configuration minimizes the number of couplers that are being used and engaged by the IDU 52. With reference to FIGS. 11, 12A, and 12B, the instrument 600 includes a housing 620, a shaft 630 extending distally from housing 620, and end effector assembly 640 extending distally from shaft 630. The end effector assembly 640 also includes first and second jaw members 642 and 644, with the jaw member 642 being movable while the jaw member 644 is stationary relative to the shaft 630. The jaw member 642 may be actuated by a coupler 650 disposed at the distal end portion of the housing 620. For a more detailed description of the components of the instrument 600 and its operation reference may be made to U.S. Pat. No. 10,722,295, filed on Jan. 20, 2016, titled “Robotic surgical assemblies and electrosurgical instruments thereof,” the entire contents of which are incorporated by reference herein.

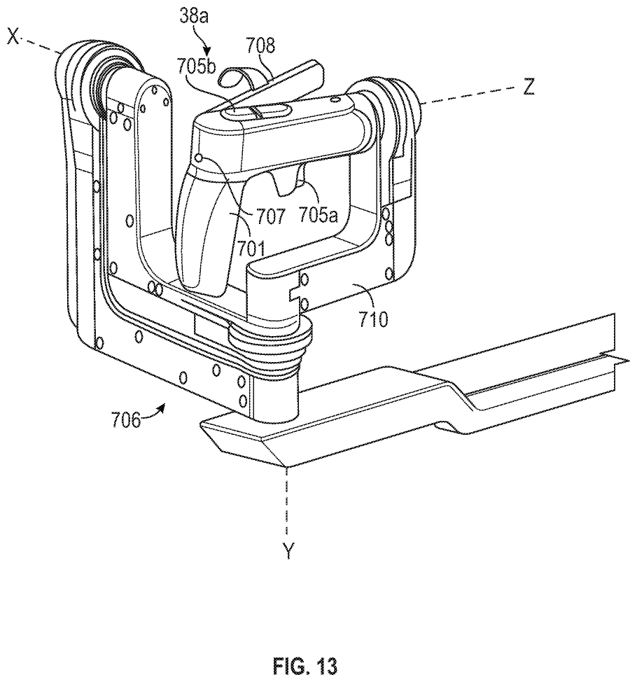

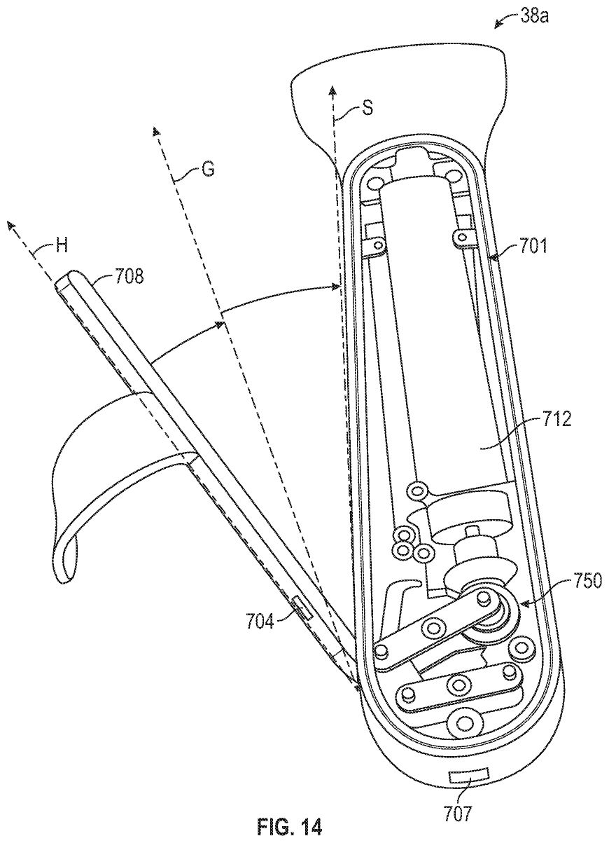

FIGS. 13 and 14 show the left-handle controller 38a, which is a mirror copy of the right-handle controller 38b. Each of the handle controllers 38a and 38b includes a handle 701 and a paddle 708 that is pivotally coupled to the handle 701 at one end (e.g., proximal) of the paddle 708. The paddle 708 is configured to control actuation, namely, opening and closing jaw members 142, 144 of the end effector assembly 140. The paddle 708 may include a finger sensor 704 (FIG. 14) configured to detect presence or movement of a finger, such as touch sensors, capacitive sensors, optical sensors, and the like. In embodiments, the finger sensor 704 may be disposed on any portion of the handle controllers 38a and 38b. Each of the handle controllers 38a and 38b may also include a trigger 705a and one or more buttons 705b for activating various functions of the instrument 50. In addition, each of the handle controllers 38a and 38b may include a gimbal assembly 706 allowing for movement and rotation of the handle controllers 38a and 38b about three axes (x, y, z). The handle controllers 38a and 38b may also include an infrared proximity sensor 707 configured to detect hand contact with a grip of the handle controllers 38a and 38b. The controller 31a of the surgeon console 30 monitors operator interactions with the handle controllers 38a and 38b and controls the instrument(s) 50 in response to operator inputs.

With reference to FIG. 14, the paddle 708 is maintained, i.e., biased, in an open position by a feedback motor 712, which receives operator mechanical input, i.e., as the motor 712 is back driven during closure of the paddle 708 toward the closed position. The paddle 708 is mechanically coupled to the motor 712 via one or more mechanical linkage components 750. Although depicted as a series of links (e.g., four bar linkage), couplers and/or actuators, any type of mechanical or electromechanical arrangement is contemplated. Moreover, and as explained below, actuation of paddle 708 relative to the actuation of the jaw members 142, 144 may be linear 1:1, non-linear 1:2 or variable during the entire range of movement or stroke of the paddle 708.

The motor 712 also provides force feedback to the paddle 708 by counteracting operator's input, i.e., the motor 712 is forward driven. In addition, the motor 712 also measures various mechanical parameters of the paddle 708, such as the applied force, angle relative to the handle 701, velocity, acceleration, etc. The angle of the paddle 708 relative to the handle 701 may be proportional to the angle between jaw members 142, 144. Thus, the paddle 708 and the jaw members 142, 144 may be fully aligned when in fully open and fully closed position and the jaw angle in between those position corresponds the paddle angle during the travel of the paddle 708.

In addition, the controller 31a also monitors individual or a new velocity of each joint of the gimbal assembly 706 as well as displacement of each of the joint of the gimbal assembly 706 and/or net displacement of the gimbal assembly 706. Details of the handle controllers 38 a and 38 b are provided in U.S. Patent Publication No. 2020/0315729, titled “Control arm assemblies for robotic surgical systems” filed on Nov. 30, 2018, the entire contents of which are incorporated by reference herein.

A feedback assembly 710 is disposed in the handle controller 38a to provide vibratory or haptic feedback to the operator. As shown, the feedback assembly 710 is configured to provide vibrational feedback at set frequencies and intervals to provide a sensation of touching. The feedback assembly 710 may include eccentric rotating mass (ERM) actuator, a linear resonant actuator (LRA), a piezoelectric actuator, or any other suitable tactile actuator configured to impart information to the operator through their sense of touch. Details of the haptic feedback mechanism are provided in U.S. Pat. No. 10,517,686, titled “Haptic feedback controls for a robotic surgical system interface” filed Apr. 13, 2018, the entire contents of which are incorporated by reference herein.

The paddle 708 is used to actuate various components of the instrument 50, e.g., open and close jaw members 142, 144. Thus, during use, the operator may apply a constant force to close the jaw members 142, 144 from fully open to fully closed configuration. To maintain full jaw closure, the operator maintains force on the paddle 708 to ensure the jaw members 142, 144 are fully closed.

With reference to FIG. 14, initial movement of paddle 708 from an unactuated position or home position “H” towards a grasp position “G” actuates one or both of the jaw members 142, 144 to grasp a vessel or tissue with a first pressure known to avoid structural damage to sensitive tissue. Pressures in the range of about 0.01 Kg/cm² to about 2 kg/cm² are contemplated. Pressures up to and including the lower range of the below-identified sealing pressure range are also contemplated. The force on the handle may be linear, e.g., the same amount of force through the entire range of motion from position “H” to position “G”, or may be non-linear, e.g., the amount of force varies within the above-identified range through the range of motion from position “H” to position “G”.

As can be appreciated, the surgeon can remotely actuate the jaw members 142, 144 to manipulate, grasp and cut (e.g., without a seal if applicable) vessels and tissue by continually moving the paddle 708 between positions “H” and position “G” without any concern of applying too much force to vessels or tissue. Vessels or tissue may also be dissected when moving the paddle 708 from position “G” to position “H”, e.g., poke and spread dissection.

Once the vessel or tissue is properly grasped, the surgeon may opt to seal the vessel or tissue between the jaw members 142, 144. In this instance the surgeon moves the paddle 708 closer to the handle 701, e.g., from position “G” to position “S” which, in turn, generates additional forces between the jaw members 142, 144 to enable sealing once the robotic sealer 10 is activated via one or more activation buttons 705a, 705b (FIG. 13). Forces within the range of about 3 kg/cm² to about 16 kg/cm² are contemplated for sealing purposes.

With reference to FIG. 15, haptic feedback may vary across multiple points “A-D” such that each paddle stroke continues from a fully open position, through a typical grasping position and to a position for sealing tissue. More particularly, point “A” position on the paddle 708 indicates a fully open position wherein the jaw members 142, 144 are fully open to orient tissue therebetween. Between point “A” and point “B”, with the paddle 708 starting to close, there is only a slight haptic force associated with the paddle 708, e.g., very little resistance on the paddle 708. Point “B” may be customizable depending on the particular instrument or based on empirical data, e.g., point “B” is configured on the paddle 708 as the point between a large vessel disposed within the jaws 142, 144 and nothing in the jaws 142, 144. In other instances, point “B” may be customizable based on the feedback from sensors or torque.

Between point “B” and point “C” the paddle 708 begins to compress a pressure control mechanism (e.g., spring 256) which will provide direct haptic feedback to the surgeon regarding pressure between jaw members 142, 144. The paddle 708 and spring relationship may be linear or exponential or may be a combination, e.g., initially linear and then exponential. In embodiments, moving the lever between points “B” and “C” may induce a pressure within the above-identified sealing range and the surgeon may opt to seal tissue based on perceived haptic feedback within this stroke range. Alternatively, a point “C” may be included within the lever stroke that definitively identifies that the jaw pressure is within the sealing range. Any further movement of the paddle 708 does not increase the pressure between the jaw members 142, 144, e.g., the pressure is offloaded by the spring (or some other pressure control mechanism (not shown)) to not over-compress the tissue. Point “D” may be included as a bottom out point of the paddle 708.

As stated above, the force feedback provided to the paddle 708 is based on the position of the paddle 708 relative to the handle 701. Thus, when the paddle 708 is closed at a sufficiently slow speed, which requires less force, the feedback is properly provided to the paddle 708 by the motor 712 according to a selected force feedback profile. However, when the paddle 708 is closed at a fast speed, which requires more force, the force feedback is insufficient. In other words, the applied force feedback, while sufficient during slow closure of the paddle 708, is insufficient during fast closure of the paddle 708. The present disclosure overcomes this issue by providing force feedback as a function of the position and velocity of the paddle 708 relative to the handle 701.

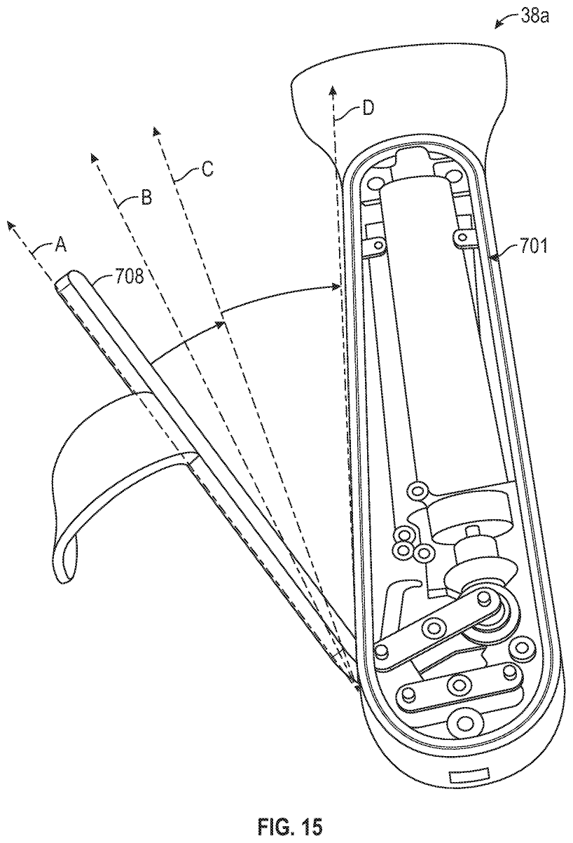

With reference to FIG. 16, a flow chart of a method for applying force feedback to the paddle 708 is presented. The method may be implemented as software instructions executable by the controller 31a of the surgeon console 30 or any other controller of the system 10. At step 800, the controller 31a detects movement of the paddle 708 and measures its velocity. The velocity of the paddle 708 is then used to select a force feedback profile based on the measured speed at step 802. The storage 22a may store a plurality of force feedback profiles for each type of instrument, which may be further tailored for the user operating the surgeon console 30.

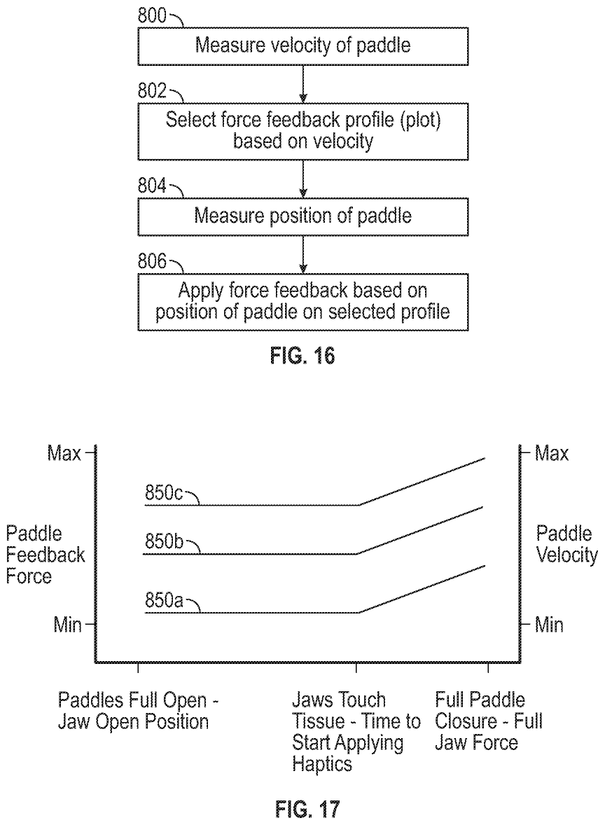

The force feedback profiles may be expressed as plots 850a-c shown in FIG. 17, in which position of the paddle 708 is expressed as a horizontal axis, the applied force is expressed as a vertical axis. Each force feedback plot 850a-c is implemented as software instructions executable by the controller 31a or any other controller of the system 10. At step 802, the controller 31a selects one of the plots 850a-c based on the measured velocity. For low velocity the controller 31a selects the plot 850a, which applies force to the paddle 708 at a low level. For medium velocity the controller 31a selects the plot 850b, which applies medium force to the paddle 708. For high velocity the controller 31a selects the plot 850c, which applies high force to the paddle 708. While only three plots 850a-c are shown, any number of force feedback may be generated, stored and selected based on the measured velocity of the paddle 708.

The plots 850a-c may have different portions corresponding to positions of the paddle 708 “H”, “G”, “S” of FIG. 14 and/or “A-D” of FIG. 15. The plots 850a-c are used by the controller 31a to apply force feedback to the paddle 708 based on the position of the paddle 708. As the paddle 708 and the jaw members 142, 144 travel from open to closed position the force applied to the paddle 708 may be increased in any suitable (e.g., linear, exponential, etc.) manner.

In embodiments, a single plot 850a may be used which is transformed based on the measured velocity along force axis. Transformation may include shifting the plot 850a along the vertical (i.e., applied force) axis. Selection of an appropriate plot 850a-c based on the velocity of the paddle 708, may be substituted by transformation of the plot 850a based on measured velocity.

Once a force feedback plot is selected, transformed, or otherwise generated, at step 804, the controller 31a measures the position, i.e., angle, of the paddle 708 and determines the force that is to be applied to the paddle 708 via the motor 712 using the selected or transformed plot 850a. At step 806, the controller 31a signals the motor 712 to apply force corresponding to the position of the paddle 708 as indicated by the plot 850c.

It is contemplated that any combination of mechanical components, electromechanical components, software, etc. may be utilized to map or correlate the various positions of the paddle 708 to the coupler 176 which, in turn, controls the relative movement of the jaw members 142, 144. Again, the relationship of the paddle 708 to the movement of the jaw members 142, 144 may be linear or non-linear depending upon a particular purpose. Moreover, the paddle 708 may be coupled to one or more mechanical, electromechanical components, e.g., components 750 (FIG. 14), or software, that are configured to vary the actuation force (either linearly or non-linearly) as the paddle 708 is actuated relative to handle 701 simulating the “feel” of the jaw members 142, 144 grasping, manipulating or sealing vessels or tissue. In other instances, a visual signal (e.g., a series of LEDs or the like or varying colors) or audible tone may be used to represent the amount of pressure being applied to the vessel or tissue during the relative movement of the paddle 708.

The force feedback on the paddle 708 is correlated to the closure pressure between the jaw members 142, 144 based on the position of the paddle 708 relative to the handle 701. In embodiments, the resistance on the paddle 708 may be related to the internal mechanical or electromechanical components 750 (FIG. 14) or may be based on software associated with one or more of the components or coupler 176. In this instance, there is no reliance on a sensor providing feedback to the components 750 or software to alter the resistance on the paddle 708.

Various visual or audible feedback may be conveyed to the surgeon relating to the amount of pressure between the jaw members 142, 144. The feedback may be a “SAFE” signal for manipulation, e.g., green light, safe tone, or may be metered in relation to the amount of pressure being applied to tissue and when a pressure is being applied to warrant sealing, e.g., “SEAL” signal. The metering of the feedback either visually or audibly provides ample feedback to the surgeon when he/she is approaching unintended pressures between the jaw members 142, 144.

In one embodiment according to the present disclosure, the handle controller 38a may be configured to switch the relative motion of the paddle 708 and the resulting closure pressure associated with the jaw members 142, 144 based on user selection, profile, or any other configuration setting. More particularly, in a first instance or with a first user selection, e.g., grasping, manipulation, cutting or dissection mode, actuation of the paddle 708 may ramp from the jaw members 142, 144 being spaced from one another (e.g., zero closure force between jaw members 142, 144) to a correlating pressure between the jaw members 142, 144 within a range of about 0.1 kg/cm² to about 2 kg/cm². Other pressure ranges for grasping, manipulation, cutting or dissection are envisioned. The relationship of the paddle 708 force to the jaw member 142, 144 closure pressure may be linear or non-linear within this range.

In a next instance or with a second or different user selection, e.g., sealing, actuation of the paddle 708 may ramp from the jaw members 142, 144 being spaced from one another to a correlating pressure between the jaw members 142, 144 within a range of about 3 kg/cm² to about 16 kg/cm². Again, the relationship of the paddle 708 force to the jaw member 142, 144 sealing pressure may be linear or non-linear within this range. Various audible or visual safety measures may be employed with the handle controller 38a or robotic surgical system 10 to alert the user of the current disposition of the paddle 708 and which pressure will be applied to the jaw members 142, 144 upon actuation thereof.

Various safety measures may be employed using one or more mechanical, electromechanical or software that would only allow a sealing pressure to be delivered when energy is being applied. For example, a slight delay may be programmed into the control software to delay activation of electrosurgical energy until a proper jaw pressure is applied between the jaw members 142, 144 via the paddle 708.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including:
an instrument drive unit; and
an instrument coupled to and actuatable by the instrument drive unit, the instrument including a first jaw member and a second jaw member, at least one of the first or second jaw members movable relative to the other of the first or second jaw members from an open jaw position to a closed jaw position; and
a surgeon console including a handle controller having:
a handle;
a paddle pivotably movable relative to the handle from an open paddle position to a paddle closed position, wherein movement of the paddle is used to control movement of at least one of the first or second jaw members; and
a feedback motor configured to provide force feedback to the paddle based on a position and a velocity of the paddle relative to the handle.

2. The surgical robotic system according to claim 1, wherein the feedback motor is configured to measure the position and the velocity of the paddle.

3. The surgical robotic system according to claim 2, further comprising:
a storage device storing a force feedback profile.

4. The surgical robotic system according to claim 3, further comprising a controller configured to:
calculate the force feedback based on the position and the velocity of the paddle from the force feedback profile; and
control the feedback motor to provide the calculated force feedback.

5. The surgical robotic system according to claim 4, wherein the controller is further configured to adjust the force feedback profile based on the velocity of the paddle.

6. The surgical robotic system according to claim 2, further comprising:
a storage device storing a plurality of force feedback profiles.

7. The surgical robotic system according to claim 6, further comprising a controller configured to:
select a force feedback profile from the plurality of force feedback profiles based on the velocity of the paddle.

8. The surgical robotic system according to claim 7, wherein the controller is further configured to:
calculate the force feedback based on the position of the paddle from the selected force feedback profile; and
control the feedback motor to provide the calculated force feedback.

9. The surgical robotic system according to claim 1, wherein the instrument further includes:
a coupler driven by a motor held by the robotic arm;
a drive rod longitudinally movable by the coupler configured to move at least one of the first or second jaw members relative to the other of the first or second jaw members; and
a spring compressed by the coupler during movement of the drive rod.

10. A method for controlling a surgical robotic system, the method comprising:
actuating an instrument coupled to and actuatable by an instrument drive unit, the instrument including a first jaw member and a second jaw member, at least one of the first or second jaw members movable relative to the other of the first or second jaw members from an open jaw position to a closed jaw position;
receiving an input from a surgeon console including a handle controller having a handle and a paddle pivotably movable relative to the handle from an open paddle position to a paddle closed position, wherein the input includes movement of the paddle;
controlling movement of at least one of the first or second jaw members by the instrument drive unit based on the input; and
providing force feedback to the paddle via a feedback motor based on a position and a velocity of the paddle relative to the handle.

11. The method according to claim 10, further comprising:
measuring the position and the velocity of the paddle through the feedback motor.

12. The method according to claim 11, further comprising:
storing a force feedback profile at a storage device.

13. The method according to claim 12, further comprising:
calculating, at a controller, the force feedback based on the position and the velocity of the paddle from the force feedback profile; and
controlling the feedback motor to provide the calculated force feedback.

14. The method according to claim 13, further comprising:
adjusting the force feedback profile based on the velocity of the paddle.

15. The method according to claim 11, further comprising:
storing a plurality of force feedback profiles at a storage device.

16. The method according to claim 15, further comprising:
selecting, at a controller, a force feedback profile from the plurality of force feedback profiles based on the velocity of the paddle.

17. The method according to claim 16, further comprising:
calculating the force feedback based on the position of the paddle from the selected force feedback profile; and
controlling the feedback motor to provide the calculated force feedback.

18. A surgical robotic system comprising:
a robotic arm including:
an instrument drive unit; and
an instrument coupled to and actuatable by the instrument drive unit, the instrument including a first jaw member and a second jaw member, at least one of the first or second jaw members movable relative to the other of the first or second jaw members from an open jaw position to a closed jaw position;
a surgeon console including a handle controller having:
a handle;
a paddle pivotably movable relative to the handle from an open paddle position to a paddle closed position, wherein movement of the paddle is used to control movement of at least one of the first or second jaw members; and
a feedback motor configured to measure a position and a velocity of the paddle relative to the handle;
a storage device storing a plurality of force feedback profiles; and
a controller configured to:
select a force feedback profile from the plurality of force feedback profiles based on the velocity of the paddle; and
control the feedback motor to provide force feedback to the paddle based on the position and the velocity of the paddle relative to the handle.

19. The surgical robotic system according to claim 18, wherein the controller is further configured to:
calculate the force feedback based on the position of the paddle from the selected force feedback profile; and
control the feedback motor to provide the calculated force feedback.

20. The surgical robotic system according to claim 18, wherein each force feedback profile includes a plurality of force feedback values each of which corresponds to a position value of the paddle.
